(19) United States
(12) Patent Application Publication
(10) Pub. No.: US 2013/0287834 A1
(43) Pub. Date: Oct. 31, 2013

(54) ANTI-ADHERENT FORMULATION INCLUDING A CATIONIC OR NONIONIC ACRYLATE CO-POLYMER

(71) Applicants: Douglas R. Hoffman, Greenville, WI (US); Corey T. Cunningham, Larsen, WI (US); Stacy A. Mundschau, Weyauwega, WI (US); David W. Koenig, Menasha, WI (US); Shaosheng Dong, Santa Clara, CA (US)

(72) Inventors: Douglas R. Hoffman, Greenville, WI (US); Corey T. Cunningham, Larsen, WI (US); Stacy A. Mundschau, Weyauwega, WI (US); David W. Koenig, Menasha, WI (US); Shaosheng Dong, Santa Clara, CA (US)

(21) Appl. No.: 13/460,313

(22) Filed: Apr. 30, 2012

(51) Int. Cl. A61K 9/70; A61P 17/00; A61K 31/78; A61K 31/80; A61K 31/79

(52) U.S. Cl. 424/443; 424/78.02

(57) ABSTRACT

Liquid formulations having anti-adherent properties are disclosed herein. The liquid anti-adherent formulation described herein acts to prevent the adherence of menses and/or fecal material to the skin in the labial and perianal regions during and after menstruation or defecation, respectively. The liquid anti-adherent formulation contains a carrier, a polymer selected from a nonionic acrylate co-polymer, cationic acrylate co-polymer, and combinations thereof. Anti-adherent formulation is found to be effective if a menses leave-on percentage as defined within the Menses Leave-On Percentage Test method described herein was less than 6.2% after two insults.

### BACKGROUND

Menses is a viscoelastic fluid composed of blood (primarily red blood cells and plasma), cervical mucus and/or tissue fragments. As menses and other non-menstrual fluids exit the vagina, they often wick along the body, causing the fluids to remain on the skin or on hair located in this region, causing the fluid to dry out and remain on the skin and/or hair. When absorbent articles are used to absorb and contain menses and other non-menstrual fluids, often the fluids do not reach the absorbent article so that the absorbent article will be able to absorb and contain the desired fluids. As a result of these fluids remaining on the skin, undesirable situations such as transfer of the fluids to undergarments, staining of undergarments, chafing, discomfort, and unwanted odors may occur.

Absorbent materials and absorbent articles are known in the art and are known to have a wide variety of uses, in particular for absorbing bodily fluids. Examples of such absorbent materials and absorbent articles include, for example; personal care products, such as disposable diapers and training pants; feminine hygiene products, such as sanitary napkins and tampons; incontinent care products, such as pads and undergarments and the like. As is mentioned above, some fluids designed to be absorbed by these articles sometimes do not reach the absorbent article, and remain deposited on skin and hair.

Numerous commercially available products exist to assist individuals in cleaning themselves in the labial and perianal regions of the body. Conventional bath tissues have been used for many years and, recently, flushable moist bath tissues have been introduced. These products may be used alone, or in combination, to effectuate cleansing of the perianal and labial regions.

Proper cleaning of skin in the perianal and vaginal regions can be difficult due to the topography of the skin in that region and the presence of hair follicles. A common problem encountered by many individuals during cleaning of these areas after bowel movements or during menstruation is the occasional sticking of fecal material or of menses to the skin in the perianal and labial areas. Additionally, because fecal material generally contains bacteria and active enzymes, the presence of this material in the perianal region after bowel movement cleanup can also result in skin irritation, redness, and even inflammation and infection for sensitive individuals. Residual menstrual fluid can support the accumulation of organic material which can persist with subsequent cleanings. These residues contain bacteria, yeast, enzymes, odor inducing agents, and microbial growth promoters. These factors can alone or in combination cause skin irritation, itching sensations, infections, as well as personal discomfort.

Based on the foregoing, it is clear that maintaining clean and healthy skin in the perianal, labial, and surrounding areas is difficult, yet important. As such, products that can improve cleaning of the skin in these regions are highly desirable, as are products which can aid in preventing menses and other fluids from adhering and sticking to the skin.

### SUMMARY

Liquid formulations having anti-adherent properties are disclosed herein. The liquid anti-adherent formulation described herein acts to prevent the adherence of menses and/or fecal material to the skin in the labial and perianal regions during and after menstruation or defecation, respectively. The liquid anti-adherent formulation contains a carrier, a polymer selected from a nonionic acrylate co-polymer, cationic acrylate co-polymer, and combinations thereof. Anti-adherent formulation was found to be effective if a menses leave-on percentage as defined within the Menses Leave-On Percentage Test method described herein was less than 6.4% after two insults.

The anti-adherent formulation may include a nonionic acrylate co-polymer having at least one methyl methacrylate monomer, and at least one monomer selected from methacryloxypropyl tris(trimethylsiloxysilane), vinyl pyrrolidinone, butyl acrylate, polydimethyl siloxane-methyl methacrylate, and combinations thereof. Desirably, the anti-adherent formulation may include from 1.0% by weight to 15.0% by weight of the nonionic polymer. More desirably, the anti-adherent formulation may include from 1.5% by weight to 10.5% by weight of the nonionic polymer.

Desirably, the nonionic acrylate co-polymer comprises about 5% to about 55% by mole of methyl methacrylate, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

The anti-adherent formulation may include a cationic acrylate co-polymer having at least one 3-acrylamidopropyl trimethylammonium chloride monomer, at least one methacryloxypropyl tris(trimethylsiloxysilane) monomer, at least one vinyl pyrrolidinone monomer, and combinations thereof. Desirably, the anti-adherent formulation may include from 1.0% by weight to 15.0% by weight of the nonionic polymer. More desirably, the anti-adherent formulation may include from 1.5% by weight to 10.5% by weight of the nonionic polymer.

Desirably, the cationic acrylate co-polymer comprises about 10% to about 40% by mole of 3-acrylamidopropyl trimethylammonium chloride, about 10% to about 55% by mole of vinyl pyrrolidinone, about 0% to about 40% by mole of butyl acrylate and about 5% to about 40% by mole of methacryloxypropyl tris(trimethylsiloxysilane).

In another desirable embodiment, the cationic acrylate co-polymer comprises about 5% to about 55% by mole of methyl methacrylate, about 0% to about 40% by mole of 3-acrylamidopropyl trimethylammonium chloride, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

Desirable embodiments may have a menses leave-on percentage as defined within the test method described herein of less than 4.0% after two insults of menses. Nonionic acrylate co-polymers included in the formulation providing this efficacy comprises about 10% to about 45% by mole of methyl methacrylate, about 0% to about 25% by mole of butyl acrylate, and about 35% to about 80% by mole of polydimethyl siloxane-methyl methacrylate.

More desirable embodiments have a menses leave-on percentage as defined within the test method described herein of less than 4.2% after four insults of menses. Nonionic acrylate co-polymers included in the formulation providing this efficacy comprises about 10% to about 45% by mole of methyl methacrylate, about 0% to about 25% by mole of butyl acrylate, and about 35% to about 80% by mole of polydimethyl siloxane-methyl methacrylate.

The anti-adherent formulation may be applied to the targeted surface either directly, in liquid form, such as by a spray bottle or similar packaging capable of delivering a liquid formulation in a relatively uniform amount over the full surface to be covered. Alternatively, the formulation may be applied to the targeted surface by a wipe on a basesheet (i.e., a “wet” wipe or wiper). Desirably, the liquid anti-adherent formulation contains at least 85% of a carrier.

These features will be described in greater detail herein. Further, it is to be understood that both the foregoing general description and the following detailed description are exemplary and are intended to provide further explanation of the invention claimed.

### DETAILED DESCRIPTION

The present disclosure will be expressed in terms of its various components, elements, constructions, configurations, arrangements, and other. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising”, and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

Formulations having anti-adherent properties are disclosed herein. The anti-adherent formulation described herein acts to prevent the adherence of menses and/or fecal material to the skin in the labial and perianal regions during and after menstruation or defecation, respectively.

The presence of the anti-adherent formulation results in a decreased amount of menstrual and/or fecal material on the skin in the labial and/or anal region during menstruation or after a bowel movement. Without being bound to a particular theory, it is believed that the anti-adherent formulation attaches to the skin through electrostatic and hydrophobic interaction with the skin and remains tightly bound thereto after deposit. When menstruation occurs, menses, which also typically attaches to skin through electrostatic and hydrophobic interactions, is not able to make the attachment to the skin as many of the binding sites are already occupied with anti-adherent formulation. Because interaction between the menses and the skin is reduced, much less menses remains attached to the skin after menstruation.

The anti-adherent may be applied to the target skin area by one of many different delivery vehicles. For example, the formulation may be applied with a wipe, including mitts and gloves, a solid stick formulation, an aerosol dispenser, a pump spray, a trigger spray, a squeeze bottle, as a foam, as a cream, as an ointment, as a salve, as a gel, as a wash, or as a lotion. In addition, absorbent articles, such as pads or pants, diapers and the like may also be used as a means to transfer the formulation to the skin. Whichever method is selected, it is desirable that the formulation be administered in an acceptable fashion to the target skin area without leaving a messy aesthetically unpleasing or uncomfortable residue on the skin. It is further desirable that the formulation be administered without direct contact with the users' or applicators' hands, which could result in a messy residue being left on the users' or applicators' hands, requiring additional clean up after application. Of the methods described above, the application with a wipe has some advantage over the other methods. For example, the wipe may be easily provided in a pouch with a disposable absorbent personal care article.

The anti-adherent formulation may be applied to the targeted surface either directly, in liquid form, such as by a spray bottle or similar packaging capable of delivering a liquid formulation in a relatively uniform amount over the full surface area to be covered. Alternatively, the formulation may be applied to the targeted surface by a carrier, such as a basesheet (i.e., a “wet” wipe or wiper). Because the formulation is liquid at room temperature, the formulation may be applied to a surface by wiping the surface with a basesheet that has been saturated with the formulation; the formulation will transfer from the basesheet to the surface. The basesheet may be formed from one or more woven materials, nonwoven materials, cellulosic materials, and combinations of such materials. More specifically, the basesheet may be formed of nonwoven fibrous sheet materials that include meltblown, spunlace, coform, air-laid, bonded-carded web materials, hydroentangled materials, and combinations of such materials. Such materials can be made of synthetic or natural fibers or a combination of such fibers. Typically, the basesheet will have a basis weight of from about 25 grams per square meter to about 120 grams per square meter and desirably from about 40 grams per square meter to about 90 grams per square meter.

The basesheet may be constructed of a coform material of polymer fibers and absorbent fibers having a basis weight of from about 45 to about 80 grams per square meter and desirably about 60 grams per square meter. Typically, such coform basesheets are constructed of a gas-formed matrix of thermoplastic polymeric meltblown fibers and cellulosic fibers. Various suitable materials may be used to provide the polymeric meltblown fibers, such as, for example, polypropylene microfibers. Alternatively, the polymeric meltblown fibers may be elastomeric polymer fibers, such as those provided by a polymer resin. For instance, VISTAMAXX elastic olefin copolymer resin designated PLTD-1810, available from ExxonMobil Corporation (Houston, Tex.) or KRATON G-2755, available from Kraton Polymers (Houston, Tex.) may be used to provide stretchable polymeric meltblown fibers for the coform basesheets. Other suitable polymeric materials or combinations thereof may alternatively be utilized as known in the art.

The coform basesheet additionally may be constructed of various absorbent cellulosic fibers, such as, for example, wood pulp fibers. Suitable commercially available cellulosic fibers for use in the coform basesheets can include, for example, NF 405, which is a chemically treated bleached southern softwood Kraft pulp, available from Weyerhaeuser Co. (Washington, D.C.); NB 416, which is a bleached southern softwood Kraft pulp, available from Weyerhaeuser Co.; CR-0056, which is a fully debonded softwood pulp, available from Bowater, Inc. (Greenville, S.C.); Golden Isles 4822 debonded softwood pulp, available from Koch Cellulose (Brunswick, Ga.); and SULPHATATE HJ, which is a chemically modified hardwood pulp, available from Rayonier, Inc. (Jessup, Ga.). The relative percentages of the polymeric meltblown fibers and cellulosic fibers in the coform basesheet may vary over a wide range depending upon the desired characteristics of the wipes. For example, the coform basesheet may have from about 10 weight percent to about 90 weight percent, desirably from about 20 weight percent to about 60 weight percent, and more desirably from about 25 weight percent to about 35 weight percent of polymeric meltblown fibers based on the dry weight of the coform basesheet.

The anti-adherent formulation may be incorporated into the basesheet in an add-on amount of from about 50% (by weight of the basesheet) to about 800% (by weight of the basesheet). More specifically, the formulations may be incorporated into the basesheet in an add-on amount of from about 200% (by weight of the basesheet) to about 600% (by weight of the basesheet) or from about 400% (by weight of the basesheet) to about 600% (by weight of the basesheet). The formulation add-on amounts may vary depending on the formulation of the basesheet.

The liquid anti-adherent formulation contains a carrier, and a polymer selected from a cationic acrylate co-polymer, nonionic acrylate co-polymer, and combinations thereof. Anti-adherent formulations were found to be effective if a menses leave-on percentage as defined within the Menses Leave-On Percentage Test method described herein was less than about 6.4% after two insults. Unexpectedly, the polymers identified were able to repel menses through multiple insults. The exact composition of menses varies from person to person, but menses generally contains between 25% to 50% water. As a result, one skilled in the art would expect that a water soluble or water dispersible polymer would be removed from the skin following one insult and absorbed into an absorbent article. However, the polymers identified clearly provide anti-adherence efficacy after multiple insults.

When coated on a surface these polymers showed greater repellency to menses than even to water, implying their mechanism for anti-adherence is not simply a hydrophobic repulsion of menses. Preferred polymers have a contact angle of between about 85 and about 110 degrees against menses. In addition, polymers that met the desired efficacy for anti-adherence following two menses insults were found to have a significantly different ratio of water contact angle to menses contact angle than polymers that did not meet the desired efficacy. The ratio of the water contact angle to the menses contact angle is greater than 0.8, and more desirably between 0.9 and 1.2.

It is important to note that the anti-adherent formulation is a liquid. A liquid formulation allows for easier application of the anti-adherent by a user. Desirably, a viscosity of the anti-adherent formulation is between 2 and 4000 centipoise.

The anti-adherent formulation may include a nonionic acrylate co-polymer having at least one methyl methacrylate monomer, and at least one monomer selected from methacryloxypropyl tris(trimethylsiloxysilane), vinyl pyrrolidinone, butyl acrylate, polydimethyl siloxane-methyl methacrylate, and combinations thereof. Desirably, the anti-adherent formulation may include from 1.0% by weight to 15.0% by weight of the nonionic polymer. More desirably, the anti-adherent formulation may include from 1.5% by weight to 10.5% by weight of the nonionic polymer.

Desirably, the nonionic acrylate co-polymer comprises about 5% to about 55% by mole of methyl methacrylate, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

The anti-adherent formulation may include a cationic acrylate co-polymer having at least one 3-acrylamidopropyl trimethylammonium chloride monomer, at least one methacryloxypropyl tris(trimethylsiloxysilane) monomer, at least one vinyl pyrrolidinone monomer, and combinations thereof. Desirably, the anti-adherent formulation may include from 1.0% by weight to 15.0% by weight of the cationic polymer. More desirably, the anti-adherent formulation may include from 1.5% by weight to 10.5% by weight of the cationic polymer.

Desirably, the cationic acrylate co-polymer comprises about 10% to about 40% by mole of 3-acrylamidopropyl trimethylammonium chloride, about 10% to about 55% by mole of vinyl pyrrolidinone, about 0% to about 40% by mole of butyl acrylate and about 5% to about 40% by mole of methacryloxypropyl tris(trimethylsiloxysilane).

In another desirable embodiment, the cationic acrylate co-polymer comprises about 5% to about 55% by mole of methyl methacrylate, about 0% to about 40% by mole of 3-acrylamidopropyl trimethylammonium chloride, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

Desirable embodiments may have a menses leave-on percentage as defined within the test method described herein of less than 4.0% after two insults of menses. Nonionic acrylate co-polymers included in the formulation providing this efficacy comprises about 10% to about 45% by mole of methyl methacrylate, about 0% to about 25% by mole of butyl acrylate, and about 35% to about 80% by mole of polydimethyl siloxane-methyl methacrylate.

More desirable embodiments have a menses leave-on percentage as defined within the test method described herein of less than 4.2% after four insults of menses. Nonionic acrylate co-polymers included in the formulation providing this efficacy comprises about 10% to about 45% by mole of methyl methacrylate, about 0% to about 25% by mole of butyl acrylate, and about 35% to about 80% by mole of polydimethyl siloxane-methyl methacrylate.

Typically, the polymers used herein have a molecular weight of between 1000 and 100,000 g/mol.

The co-polymers may be synthesized using a typical acrylate co-polymer synthesis. An exemplary procedure for synthesizing the co-polymers is adapted from the synthetic procedures described in the journal article by Charles L. McCormick and Andrew B. Lowe entitled “Aqueous RAFT polymerization: recent developments in synthesis of functional water-soluble (co)polymers with controlled structure” Accounts of Chemical Research, 2004, 37, 312-325 and the journal article by Yulia A. Vasilieva, David B. Thomas, Charles W. Scales, and Charles L. McCormick entitled “Direct controlled polymerization of a cationic methacrylamido monomer in aqueous media via the RAFT process” Macromolecules, 2004, 37, 2728-2737.

As described above, the anti-adherent formulation also includes at least about 85% by weight of a carrier. Non-limiting examples of suitable carrier materials include water; glycols such as propylene glycol, butylene glycol, and ethoxydiglycol; lower chain alcohols such as ethanol and isopropanol; glycerin and glycerin derivatives; natural oils such as jojoba oil and sunflower oil; synthetic oils such as mineral oil; silicone derivatives such as cyclomethicone, and other pharmaceutically acceptable carrier materials. As will be recognized by one skilled in the art, the relative amounts of carrier material and other components in the formulations that can be used to formulate the formulation will be dictated by the nature of the formulation. The levels can be determined by routine experimentation in view of the disclosure provided herein.

The liquid anti-adherent formulation desirably contains water. The liquid anti-adherent formulation can suitably contain water in an amount of from about 50% by weight of the formulation to about 98.5% by weight of the formulation, and more desirably from about 60% by weight of the formulation to about 98.5% by weight of the formulation. For instance, where the formulation is used in connection with a wet wipe, the formulation can suitably contain water in an amount of from about 75% by weight of the formulation to about 98.5% by weight of the formulation.

Additionally, the liquid anti-adherent formulation desirably contains a polyol to help stabilize the formulation. Desirably, this is propylene glycol, butylene glycol, or glycerin. The liquid anti-adherent formulations can suitably contain a polyol in an amount of from about 0.5% by weight of the formulation to about 5.0% by weight of the formulation.

When the anti-adherent formulation is used in connection with a wet wipe, the anti-adherent formulation may also include an anti-foaming agent. Desirably, the anti-foaming agent may be polydimethyl silicone emulsion such as SAG* 710 Silicone Antifoam Emulsion commercially available from Union Carbide Corporation (Danbury, Conn.). The liquid anti-adherent formulations can suitably contain the anti-foaming agent in an amount of from about 0.1% by weight of the formulation to about 0.5% by weight of the formulation.

Additionally, the anti-adherent formulation may include a compatible surfactant. The surfactant is selected from cationic surfactants, non-ionic surfactants, zwitterionic surfactants, and combinations thereof. The anti-adherent formulation may suitably include one or more compatible surfactants in an amount of from about 0.01% by weight of the formulation to about 10% by weight of the formulation.

As described above, the surfactant may be a nonionic surfactant. Nonionic surfactants typically have a hydrophobic base, such as a long chain alkyl group or an alkylated aryl group, and a hydrophilic chain comprising a certain number (e.g., 1 to about 30) of ethoxy and/or propoxy moieties. Examples of some classes of nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈₋₁₈) acids, condensation products of ethylene oxide with long chain amines or amides, condensation products of ethylene oxide with alcohols, and combinations thereof.

Various specific examples of suitable nonionic surfactants for use in the anti-adherent formulation include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, ethoxylated fatty (C1-22) alcohol, including 3 to 20 ethylene oxide moieties, polyoxyethylene-20 isohexadecyl ether, polyoxyethylene-23 glycerol laurate, PEG-80 sorbitan laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG-12 dioleate, PEG-8 dioleate, and combinations thereof.

Additional nonionic surfactants that can be used include water soluble alcohol ethylene oxide condensates, such as the condensation products of a secondary aliphatic alcohol containing between about 8 to about 18 carbon atoms in a straight or branched chain configuration condensed with between about 5 to about 30 moles of ethylene oxide. Such nonionic surfactants are commercially available under the trade name Tergitol from Union Carbide Corp. (Danbury, Conn.). Specific examples of such commercially available nonionic surfactants of the foregoing type are C₁₁₋₁₅ secondary alkanols condensed with either 9 moles of ethylene oxide (Tergitol 15-S-9) or 12 moles of ethylene oxide (Tergitol 15-S-12).

Other suitable nonionic surfactants include the polyethylene oxide condensates of one mole of alkyl phenol containing from about 8 to 18 carbon atoms in a straight or branched chain alkyl group with about 5 to 30 moles of ethylene oxide. Specific examples of alkyl phenol ethoxylates include nonyl condensed with about 9.5 moles of ethylene oxide per mole of nonyl phenol, dinonyl phenol condensed with about 12 moles of ethylene oxide per mole of phenol, dinonyl phenol condensed with about 15 moles of ethylene oxide per mole of phenol and diisoctylphenol condensed with about 15 moles of ethylene oxide per mole of phenol. Commercially available nonionic surfactants of this type include Igepal CO-630 (a nonyl phenol ethoxylate) from ISP Corpation (Wayne, N.J.). Suitable non-ionic ethoxylated octyl and nonyl phenols include those having from about 7 to about 13 ethoxy units. Such compounds are commercially available under the trade name Triton X from Union Carbide Corporation (Danbury, Conn.).

Alkyl polyglycosides may also be used as a nonionic surfactant in the anti-adherent formulation. Suitable alkyl polyglycosides are known nonionic surfactants that are alkaline and electrolyte stable. Alkyl mono and polyglycosides are prepared generally by reacting a monosaccharide, or a compound hydrolyzable to a monosaccharide with an alcohol such as a fatty alcohol in an acid medium.

Suitable zwitterionic surfactants for use in the anti-adherent formulation include, for example, alkyl amine oxides, silicone amine oxides, and combinations thereof. Various specific zwitterionic surfactants for use in the anti-adherent formulation include, for example, 4-[N,N-di(2-hydroxyethyl)-N-octadecylammonio]-butane-1-carboxylate, 5-[S-3-hydroxypropyl-S-hexadecylsulfonio]-3-hydroxypentane-1-sulfate, 3-[P,P-diethyl-P-3,6,9-trioxatetradexopcylphosphonio]-2-hydroxypropane-1-phosphate, 3-(N,N-dipropyl-N-3-dodecoxy-2-hydroxypropylammonio]-propane-1-phosphonate, 3-(N,N-dimethyl-N-hexadecylammonio)propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, and combinations thereof.

Suitable cationic surfactants for use in the anti-adherent formulation include, for example, alkyl ammonium salts, polymeric ammonium salts, alkyl pyridinium salts, aryl ammonium salts, alkyl aryl ammonium salts, silicone quaternary ammonium compounds, and combinations thereof. Specific examples of cationic surfactants include behenyltrimonium chloride, stearlkonium chloride, distearalkonium chloride, chlorohexidine digluconate, polyhexamethylene biguanide (PHMB), cetyl pyridinium chloride, benzethonium chloride, benzalkoniumchloride, and combinations thereof.

In addition to the components described herein, the anti-adherent formulation may also include a pH adjuster, fragrance, preservative, dye, corrosion inhibitor, builder, cleansing solvent, and other components known to be useful in personal care formulations.

As described above, the anti-adherent formulation provides resistance from menses and/or fecal material sticking to the hair and skin of a user. Thus, when the anti-adherent formulation is placed on a surface such as skin, and menses or fecal material then contacts the skin, less of the menses or fecal material remains on the skin. The menses leave-on percentage is defined as calculated per the test method described below. Desirably, the menses leave-on percentage is less than 6.4% after two insults with simulated menses material.

### Test Method

### Menses Leave-On Percentage

The Menses Leave-On Percentage was calculated using simulated skin and simulated menses. Vitro-Skin® samples commercially available from IMS Testing Group (Portland, Me.) were prepared by cutting to a dimension of 4×4 cm. The Vitro-Skin® samples were adhered to a 5×5 cm glass slide. 100 mg of each prospective anti-adherent formulation is added to Vitro-Skin® and spread evenly across the surface using a glass rod. For applications using wipes, a 15.2×18.8 cm wet wipe constructed of hydroknit wetted with a prospective anti-adherent formulation was folded in half, four times. Holding the glass slide with one hand and the wet wipe in the other the wipe was pressed to the Vitro-Skin® using firm, even pressure when wiping across the top half of the skin. Using the same technique, the bottom half of the skin was wiped. This was repeated so that the Vitro-Skin(®) was wiped 4 times on the top and bottom half of the Vitro-Skin®. The Vitro-Skin® was allowed to dry for a set amount of time. The Vitro-Skin® was allowed to dry for at least 60 seconds following treatment application. Untreated Vitro-Skin® was used as a negative control.

The Vitro-Skin®, Kotex® UltraThin (Regular) feminine pads commercially available from Kimberly-Clark Corporation (Neenah, Wis.), and menses simulant were placed in a humidity chamber at 85% humidity and 32° C. and allowed to acclimate for 1 hour. After 1 hour the mass of each Vitro-Skin® and feminine pad was recorded. Then, 1000 mg of menses simulant was applied to the Vitro-Skin® and allowed a contact time of 60 seconds. A feminine pad was placed on the Vitro-Skin® and a 409 g mass weight (10 cm×15 cm) was placed onto the feminine pad. The feminine pad and weight were left on the Vitro-Skin® for 60 seconds. In the case of multiple insults, after the initial 60 seconds, 1000 mg of menses simulant was applied to the Vitro-Skin® and allowed a contact time of 60 seconds before the feminine pad and weight were placed on the Vitro-Skin® for an additional 60 seconds. This was repeated for the desired amount of insults. For example, in the case of 4 insults, this procedure was repeated 4 times.

After the desired amount of insults and application of the feminine pad, the feminine pad and weight were removed and the mass of the Vitro-Skin® and feminine pad was recorded. The percent menses left behind on the skin and feminine pad was calculated by dividing the mass left behind on Vitro-Skin® by the sum of the mass left behind on the pad and the mass left behind on Vitro-Skin®. The mass left behind on Vitro-Skin® and pads was determined by subtracting final mass values from baseline values.

### Contact Angle

Contact angle values were determined using a Krüss DSA100 Drop Shape Analyzer. Briefly, individual glass slides were coated with each respective polymer and a drop of water or menses simulant was added. The drop was allowed to come to rest then images were captured and analyzed to quantify the contact angle.

### EXAMPLES

### Example 1

In this example, exemplary anti-adherent polymers were prepared. Each of the polymers was tested with two insults of menses simulant as described in the Menses Leave-On Percentage Test described above to determine the menses leave-on percentage. The values in Table 1 represent the mole fractions of the individual monomers forming the cationic polymer.

TABLE 1
Exemplary polymers

| Example | | | | | | |
|---|---|---|---|---|---|---|
| Polymer | APTAC | VP | MMA | BA | TRIS | PDMS-MMA |
| 1 | 0 | 0 | 0 | 55 | 25 | 20 |
| 2 | 0 | 0 | 0 | 50 | 35 | 15 |
| 3 | 0 | 40 | 0 | 50 | 10 | 0 |
| 4 | 0 | 0 | 0 | 50 | 10 | 40 |
| 5 | 0 | 0 | 0 | 50 | 30 | 20 |
| 6 | 0 | 20 | 0 | 50 | 30 | 0 |
| 7 | 0 | 0 | 0 | 45 | 35 | 20 |
| 8 | 0 | 0 | 0 | 45 | 15 | 40 |
| 9 | 0 | 0 | 0 | 45 | 5 | 50 |
| 10 | 0 | 15 | 0 | 42.5 | 42.5 | 0 |
| 11 | 0 | 0 | 0 | 42.5 | 42.5 | 15 |
| 12 | 0 | 40 | 0 | 40 | 20 | 0 |
| 13 | 0 | 0 | 0 | 40 | 40 | 20 |
| 14 | 0 | 20 | 0 | 40 | 40 | 0 |
| 15 | 0 | 0 | 0 | 40 | 20 | 40 |
| 16 | 0 | 0 | 0 | 40 | 10 | 50 |
| 17 | 0 | 25 | 0 | 37.5 | 37.5 | 0 |
| 18 | 0 | 0 | 0 | 35 | 15 | 50 |
| 19 | 0 | 0 | 0 | 35 | 5 | 60 |
| 20 | 0 | 0 | 0 | 35 | 45 | 20 |
| 21 | 0 | 0 | 0 | 35 | 25 | 40 |
| 22 | 0 | 60 | 0 | 35 | 5 | 0 |
| 23 | 0 | 0 | 0 | 32.5 | 7.5 | 60 |
| 24 | 0 | 35 | 0 | 32.5 | 32.5 | 0 |
| 25 | 0 | 0 | 0 | 30 | 10 | 60 |
| 26 | 0 | 0 | 0 | 30 | 20 | 50 |
| 27 | 0 | 60 | 0 | 30 | 10 | 0 |
| 28 | 0 | 40 | 0 | 30 | 30 | 0 |
| 29 | 0 | 45 | 0 | 27.5 | 27.5 | 0 |
| 30 | 0 | 0 | 0 | 25 | 25 | 50 |
| 31 | 0 | 0 | 0 | 25 | 15 | 60 |
| 32 | 0 | 55 | 0 | 22.5 | 22.5 | 0 |
| 33 | 0 | 0 | 0 | 20 | 0 | 80 |
| 34 | 0 | 80 | 0 | 20 | 0 | 0 |
| 35 | 0 | 0 | 0 | 20 | 20 | 60 |
| 36 | 0 | 60 | 0 | 20 | 20 | 0 |
| 37 | 0 | 0 | 0 | 17 | 3 | 80 |
| 38 | 0 | 0 | 0 | 16 | 4 | 80 |
| 39 | 0 | 0 | 0 | 15 | 5 | 80 |
| 40 | 0 | 80 | 0 | 15 | 5 | 0 |
| 41 | 0 | 0 | 0 | 14 | 6 | 80 |
| 42 | 0 | 0 | 0 | 12.5 | 7.5 | 80 |
| 43 | 0 | 80 | 0 | 12.5 | 7.5 | 0 |
| 44 | 0 | 0 | 0 | 12 | 8 | 80 |
| 45 | 40 | 10 | 40 | 10 | 0 | 0 |
| 46 | 40 | 10 | 45 | 5 | 0 | 0 |
| 47 | 10 | 40 | 10 | 0 | 40 | 0 |
| 48 | 20 | 30 | 20 | 0 | 30 | 0 |
| 49 | 15 | 30 | 15 | 0 | 40 | 0 |
| 50 | 15 | 30 | 15 | 0 | 40 | 0 |
| 51 | 10 | 40 | 10 | 0 | 40 | 0 |
| 52 | 15 | 40 | 15 | 0 | 30 | 0 |
| 53 | 15 | 40 | 15 | 0 | 30 | 0 |
| 54 | 40 | 5 | 55 | 0 | 0 | 0 |
| 55 | 30 | 30 | 30 | 0 | 10 | 0 |
| 56 | 20 | 40 | 20 | 0 | 20 | 0 |
| 57 | 0 | 0 | 0 | 14 | 6 | 80 |
| 58 | 0 | 0 | 0 | 50 | 35 | 15 |
| 59 | 0 | 0 | 0 | 15 | 5 | 80 |
| 60 | 0 | 0 | 0 | 35 | 5 | 60 |
| 61 | 0 | 0 | 0 | 17 | 3 | 80 |
| 62 | 0 | 0 | 0 | 20 | 0 | 80 |
| 63 | 0 | 0 | 0 | 35 | 15 | 50 |
| 64 | 0 | 0 | 0 | 16 | 4 | 80 |
| 65 | 0 | 0 | 0 | 30 | 10 | 60 |

Legend:
APTAC = 3-acrylamidopropyl trimethylammonium chloride
VP = vinyl pyrrolidinone
MMA = methyl methacrylate
BA = butyl acrylate
TRIS = methacryloxypropyl tris(trimethylsiloxysilane)
PDMA-MMA = polydimethyl siloxane-methyl methacrylate

Table 2 illustrates the the menses leave-on percentage for each polymer, the contact angle with water, the contact angle with menses, and the ratio of contact angles.

TABLE 2
Experimental Data

| | Menses | | | |
|---|---|---|---|---|
| | Leave-On | Contact | Contact | Contact Angle with |
| Example | % after | Angle with | Angle with | Water/Contact |
| Polymer | 2 insults | Water | Menses | Angle with Menses |
| 1 | 5.4 | 99.9 | 96.9 | 1.03 |
| 2 | 3.9 | 93.5 | 97.7 | 0.96 |
| 3 | 4.0 | 95.1 | 100.2 | 0.95 |
| 4 | 5.0 | 103.8 | 101.7 | 1.02 |
| 5 | 5.0 | 98.2 | 95.2 | 1.03 |
| 6 | 5.5 | 90.9 | 97.7 | 0.93 |
| 7 | 4.1 | 99.6 | 98.6 | 1.01 |
| 8 | 4.5 | 103.9 | 96.5 | 1.08 |
| 9 | 5.7 | 103.4 | 99.5 | 1.04 |
| 10 | 4.6 | 96.4 | 94.2 | 1.02 |
| 11 | 4.7 | 88.9 | 91.0 | 0.98 |
| 12 | 4.0 | 95.7 | 100.0 | 0.96 |
| 13 | 4.4 | 91.3 | 100.8 | 0.91 |
| 14 | 4.7 | 87.9 | 92.1 | 0.95 |
| 15 | 5.3 | 104.4 | 95.0 | 1.10 |
| 16 | 5.6 | 104.7 | 98.7 | 1.06 |
| 17 | 5.8 | 94.6 | 101.9 | 0.93 |
| 18 | 2.9 | 103.9 | 92.5 | 1.12 |
| 19 | 3.4 | 105.7 | 96.8 | 1.09 |
| 20 | 4.4 | 100.7 | 100.3 | 1.00 |
| 21 | 4.6 | 100.3 | 97.3 | 1.03 |
| 22 | 5.3 | 97.4 | 99.4 | 0.98 |
| 23 | 4.1 | 104.1 | 93.9 | 1.11 |
| 24 | 5.9 | 95.8 | 101.4 | 0.94 |
| 25 | 2.4 | 105.4 | 92.3 | 1.14 |
| 26 | 4.1 | 96.3 | 102.0 | 0.94 |
| 27 | 4.2 | 94.8 | 99.8 | 0.95 |
| 28 | 4.2 | 94.0 | 100.3 | 0.94 |
| 29 | 4.6 | 94.8 | 103.2 | 0.92 |
| 30 | 5.4 | 100.5 | 102.5 | 0.98 |
| 31 | 5.5 | 106.4 | 97.0 | 1.10 |
| 32 | 4.9 | 96.5 | 106.7 | 0.90 |
| 33 | 3.0 | 89.8 | 90.0 | 1.00 |
| 34 | 5.5 | 95.8 | 101.5 | 0.94 |
| 35 | 6.1 | 100.8 | 105.0 | 0.96 |
| 36 | 6.3 | 91.5 | 102.7 | 0.89 |
| 37 | 3.4 | 103.1 | 90.7 | 1.14 |
| 38 | 2.8 | 89.6 | 88.3 | 1.01 |
| 39 | 3.5 | 97.5 | 90.4 | 1.08 |
| 40 | 5.9 | 95.2 | 99.9 | 0.95 |
| 41 | 4.0 | 95.3 | 84.0 | 1.13 |
| 42 | 4.9 | 94.1 | 86.8 | 1.08 |
| 43 | 6.0 | 95.7 | 101.8 | 0.94 |
| 44 | 5.8 | 84.5 | 85.5 | 0.99 |
| 45 | 5.5 | 33.8 | 42.0 | 0.80 |
| 46 | 6.3 | 34.2 | 48.2 | 0.71 |
| 47 | 4.4 | 72.1 | 84.8 | 0.85 |
| 48 | 5.0 | 45.6 | 79.3 | 0.58 |
| 49 | 5.4 | 44.9 | 73.3 | 0.61 |
| 50 | 5.4 | 39.3 | 65.9 | 0.60 |
| 51 | 5.4 | 71.3 | 86.5 | 0.82 |
| 52 | 5.7 | 61.0 | 80.3 | 0.76 |
| 53 | 6.0 | 57.8 | 88.7 | 0.65 |
| 54 | 6.2 | 29.0 | 30.4 | 0.95 |
| 55 | 6.3 | 59.1 | 78.5 | 0.75 |
| 56 | 6.3 | 73.7 | 85.5 | 0.86 |
| 57 | 4.0 | 95.3 | 84.0 | 1.13 |
| 58 | 3.9 | 93.5 | 97.7 | 0.96 |
| 59 | 3.5 | 97.5 | 90.4 | 1.08 |
| 60 | 3.4 | 105.7 | 96.8 | 1.09 |
| 61 | 3.4 | 103.1 | 90.7 | 1.14 |
| 62 | 3.0 | 89.8 | 90.0 | 1.00 |
| 63 | 2.9 | 103.9 | 92.5 | 1.12 |
| 64 | 2.8 | 89.6 | 88.3 | 1.01 |
| 65 | 2.4 | 105.4 | 92.3 | 1.14 |

Each of the desirable polymers illustrated in Table 2 demonstrated an efficacy of less than 6.4% menses leave-on after two insults. More desirable Exemplary Polymers 57-65 demonstrated an efficacy of less than 4.0% menses leave-on after two insults. In addition, desirable Exemplary Polymers demonstrated a ratio of a contact angle with water to a contact angle of menses to greater than 0.8.

### Example 2

In this example, exemplary anti-adherent polymers were prepared and tested with four insults of menses simulant as described in the Menses Leave-On Percentage Test described above to determine the menses leave-on percentage. Table 3 illustrates the exemplary formulation that corresponds with the number in Table 1 and the menses leave-on percentage for each polymer. Each of the desirable polymers illustrated in Table 3 demonstrated an efficacy of less than 4.6% menses leave-on after four insults.

TABLE 3
Exemplary Efficacious Formulations after Four Insults

| Example | Menses Leave-On % |
|---|---|
| Polymer | after 4 insults |
| 16 | 3.4 |
| 19 | 3.8 |
| 59 | 2.1 |
| 62 | 3.0 |
| 63 | 3.1 |
| 64 | 2.6 |
| 65 | 3.1 |

While the anti-adherent formulation has been described in detail with respect to specific aspects thereof, it will be appreciated that those skilled in the art, upon attaining an understanding of the foregoing, may readily conceive of alterations to, variations of and equivalents to these formulations. Accordingly, the scope of the present invention should be assessed as that of the claims and any equivalents thereto.

1. A liquid anti-adherent formulation having anti-adherent activity comprising:
a carrier,
from about 1.5% to about 15% by weight of nonionic acrylate co-polymer wherein the nonionic acrylate co-polymer comprises about 5% to about 55% by mole of methyl methacrylate, about 0% to about 45% by mole of vinyl pyrrolidinone, about 0% to about 45% by mole of butyl acrylate, about 0% to about 80% by mole of methacryloxypropyl tris(trimethylsiloxysilane) methacryloxypropyl tris(trimethylsiloxy)silane and about 0% to about 80% by mole of polydimethyl siloxane-methyl methacrylate,
wherein liquid anti-adherent formulation has a menses leave-on percentage of less than about 6.4% after two insults.

2. (canceled)

3. The liquid anti-adherent formulation of claim 1 wherein the formulation has a menses leave-on of less than about 4.0% after two insults.

4. The liquid anti-adherent formulation of claim 1 wherein the nonionic acrylate co-polymer comprises about 10% to about 55% by mole of methyl methacrylate, about 0% to about 40% by mole of butyl acrylate, and about 10% to about 80% by mole of polydimethyl siloxane-methyl methacrylate.

5. The liquid anti-adherent formulation of claim 3 wherein the formulation has a menses leave-on percentage of less than about 4.6% after four insults.

6. The liquid anti-adherent formulation of claim 5 wherein the nonionic acrylate co-polymer comprises about 10% to about 45% by mole of methyl methacrylate, about 0% to about 25% by mole of butyl acrylate, and about 35% to about 80% by mole of polydimethyl siloxane-methyl methacrylate.

7. The liquid anti-adherent formulation of claim 1 wherein the carrier comprises water and alcohol, and wherein the anti-adherent formulation contains between about 85% and about 99% by weight of the carrier.

8. The liquid anti-adherent formulation of claim 1 wherein the viscosity of the anti-adherent formulation is between about 2 and about 4000 centipoise.

9. The liquid anti-adherent formulation of claim 1 wherein the ratio of a water contact angle to a menses contact angle is greater than 0.8.

10. A liquid anti-adherent formulation having anti-adherent activity comprising:
a carrier,
from about 1.5% to about 15.0% by weight of a cationic acrylate co-polymer having at least one 3-acrylamidopropyl trimethylammonium chloride monomer, at least one methacryloxypropyl tris(trimethylsiloxysilane) monomer, at least one vinyl pyrrolidinone monomer, and combinations thereof,
wherein liquid anti-adherent formulation has a menses leave-on percentage of less than about 6.4% after two insults.

11. The liquid anti-adherent formulation of claim 10 wherein the cationic acrylate co-polymer further comprises at least one butyl acrylate monomer.

12. The liquid anti-adherent formulation of claim 10 wherein the cationic acrylate co-polymer comprises about 10% to about 40% by mole of 3-acrylamidopropyl trimethylammonium chloride, about 10% to about 55% by mole of vinyl pyrrolidinone, about 0% to about 40% by mole of butyl acrylate, and about 5% to about 40% by mole of methacryloxypropyl tris(trimethylsiloxysilane).

13. The liquid anti-adherent formulation of claim 10 wherein the cationic acrylate co-polymer comprises about 5% to about 55% by mole of methyl methacrylate, about 0% to about 40% by mole of 3-acrylamidopropyl trimethylammonium chloride, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

14. The anti-adherent formulation of claim 10 wherein the carrier comprises water and alcohol, and wherein the anti-adherent formulation contains between about 85% and about 99% by weight of the carrier.

15. The anti-adherent formulation of claim 10 wherein the viscosity of the anti-adherent formulation is between about 2 and about 4000 centipoise.

16. A wet wipe that imparts a perceivable aesthetic benefit to skin, the wipe comprising:
a wipe substrate; and
a liquid formulation containing:
at least about 85% of a carrier, and
from 1.5% to about 15.0% by weight of an anti-adherent component selected from a nonionic acrylate co-polymer, a nonionic acrylate co-polymer, and combinations thereof,
wherein the nonionic acrylate co-polymer comprises at least one methyl methacrylate monomer, and at least one monomer selected from methacryloxypropyl tris(trimethylsiloxysilane), vinyl pyrrolidinone, butyl acrylate, polydimethyl siloxane-methyl methacrylate, and combinations thereof,
wherein the nonionic acrylate co-polymer comprises at least one 3-acrylamidopropyl trimethylammonium chloride monomer, at least one methacryloxypropyl tris(trimethylsiloxysilane) monomer, at least one vinyl pyrrolidinone monomer, and combinations thereof,

17. The wet wipe of claim 16 wherein the nonionic acrylate co-polymer comprises about 5% to about 55% by mole of methyl methacrylate, about 0% to about 45% by mole of vinyl pyrrolidinone, about 0% to about 45% by mole of butyl acrylate, about 0% to about 80% by mole of methacryloxypropyl tris(trimethylsiloxysilane), and about 0% to about 80% by mole of polydimethyl siloxane-methyl methacrylate.

18. The wet wipe of claim 16 wherein the formulation has a menses leave-on of less than about 4.0% after two insults.

19. The wet wipe of claim 18 wherein the nonionic acrylate co-polymer comprises about 10% to about 55% by mole of methyl methacrylate, about 0% to about 40% by mole of butyl acrylate, and about 10% to about 80% by mole of polydimethyl siloxane-methyl methacrylate.

20. The wet wipe of claim 19 wherein the formulation has a menses leave-on percentage of less than about 4.6% after four insults.

21. The wet wipe of claim 20 wherein the nonionic acrylate co-polymer comprises about 10% to about 45% by mole of methyl methacrylate, about 0% to about 25% by mole of butyl acrylate, and about 35% to about 80% by mole of polydimethyl siloxane-methyl methacrylate.

22. The wet wipe of claim 16 wherein the carrier comprises water and alcohol, and wherein the anti-adherent formulation contains between about 85% and about 99% by weight of the carrier.

23. The wet wipe of claim 16 wherein the viscosity of the anti-adherent formulation is between about 2 and about 4000 centipoise.

24. The wet wipe of claim 16 wherein the ratio of a water contact angle to a menses contact angle is greater than 0.8.

25. The wet wipe of claim 16 wherein the cationic acrylate co-polymer further comprises at least one butyl acrylate monomer.

26. The wet wipe of claim 25 wherein the cationic acrylate co-polymer comprises about 10% to about 40% by mole of 3-acrylamidopropyl trimethylammonium chloride, about 10% to about 55% by mole of vinyl pyrrolidinone, about 0% to about 40% by mole of butyl acrylate, and about 5% to about 40% by mole of methacryloxypropyl tris(trimethylsiloxysilane).
